(19) United States
(12) Patent Application Publication
(10) Pub. No.: US 2022/0230721 A1
(43) Pub. Date: Jul. 21, 2022

(54) MEDICAL INFORMATION PROCESSING APPARATUS

(71) Applicant: CANON MEDICAL SYSTEMS CORPORATION, Tochigi (JP)

(72) Inventors: Kohei SHINOHARA, Nasushiobara (JP); Hikaru FUTAMI, Nasushiobara (JP); Shuhei BANNAE, Utsunomiya (JP); Hisaaki OOSAKO, Utsunomiya (JP)

(73) Assignee: CANON MEDICAL SYSTEMS CORPORATION, Tochigi (JP)

(21) Appl. No.: 17/646,387

(22) Filed: Dec. 29, 2021

(30) Foreign Application Priority Data
Jan. 21, 2021 (JP) 2021-007745

(51) Int. Cl. G16H 20/10; G16H 10/20; G16H 10/60; G16B 50/00

(52) U.S. Cl. CPC G16H 20/10; G16B 50/00; G16H 10/60; G16H 10/20

(57) ABSTRACT

A medical information processing apparatus according to the present embodiment includes a processing circuitry. The processing circuitry is configured to acquire gene therapy information in which a candidate agent that is a therapeutic agent for a gene mutation related to a disease of a patient, an effect of the candidate agent, and ancillary information on the candidate agent are associated with one another. The processing circuitry is configured to determine a priority order of the candidate agent based on the gene therapy information and patient information on the patient. The processing circuitry is configured to present the priority order in association with information representing a relationship between genes.

### CROSS-REFERENCE TO RELATED APPLICATIONS

This application is based upon and claims the benefit of priority from Japanese Patent Application No. 2021-007745, filed on Jan. 21, 2021; the entire contents of which is incorporated herein by reference.

### FIELD

Embodiments described herein relate generally to a medical information processing apparatus.

### BACKGROUND

For example, in a hospital, as a result of testing genes of a patient, information on gene mutations related to diseases of the patient is acquired to determine therapeutic agents suitable for the patient. In this case, for example, a doctor who operates a terminal in the hospital reads out information stored in a gene database and determines therapeutic agents for the gene mutations related to the diseases of the patient as candidate agents in the order of the higher therapeutic effect. The doctor proposes the determined candidate agents to the patient as therapeutic agents suitable for the patient. However, in a case where the candidate agents are determined in consideration of only the therapeutic effect, the patient may not undergo treatment with the candidate agents due to various circumstances of the patient.

### BRIEF DESCRIPTION OF THE DRAWINGS

FIG. 1 is a diagram illustrating an example of a configuration of a medical information processing system including a medical information processing apparatus according to a first embodiment;

FIG. 2 is a diagram for explaining each processing function of the medical information processing apparatus according to the first embodiment;

FIG. 3 is a diagram illustrating an example of a genetic map according to the first embodiment;

FIG. 4 is a diagram illustrating processing executed by the medical information processing apparatus according to the first embodiment;

FIG. 5A is a flowchart illustrating a processing procedure executed by the medical information processing apparatus according to the first embodiment;

FIG. 5B is a flowchart illustrating a processing procedure executed by the medical information processing apparatus according to the first embodiment;

FIG. 5C is a flowchart illustrating a processing procedure executed by the medical information processing apparatus according to the first embodiment;

FIG. 6 is a diagram illustrating processing executed by the medical information processing apparatus according to a second embodiment;

FIG. 7 is a diagram illustrating processing executed by the medical information processing apparatus according to a third embodiment;

FIG. 8 is a diagram illustrating processing executed by the medical information processing apparatus according to a fourth embodiment; and

FIG. 9 is a diagram illustrating the processing executed by the medical information processing apparatus according to the fourth embodiment.

### DETAILED DESCRIPTION

A medical information processing apparatus according to the present embodiment is provided with a processing circuitry. The processing circuitry is configured to acquire gene therapy information in which a candidate agent that is a therapeutic agent for a gene mutation related to a disease of a patient, an effect of the candidate agent, and ancillary information on the candidate agent are associated with one another. The processing circuitry is configured to determine a priority order of the candidate agent based on the gene therapy information and patient information on the patient. The processing circuitry is configured to present the priority order in association with information representing a relationship between genes.

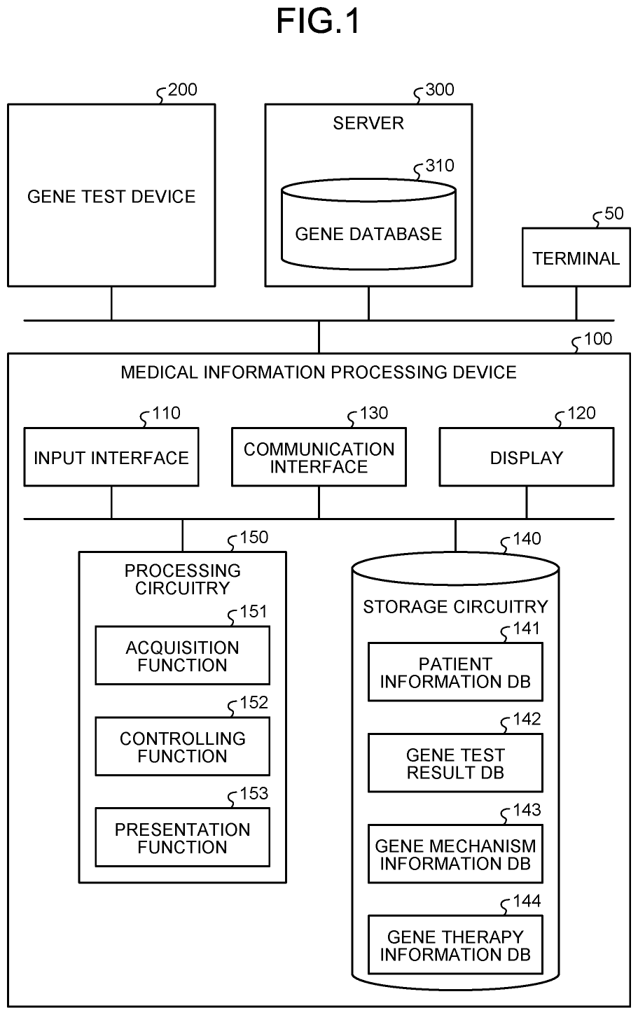

Hereinafter, embodiments of the medical information processing apparatus will be described in detail with reference to the accompanying drawings. Hereinafter, a medical information processing system including the medical information processing apparatus will be described with examples. In the medical information processing system illustrated in FIG. 1, each device is illustrated as only one, but in the practical use, a plurality of the devices can be included.

### First Embodiment

FIG. 1 is a diagram illustrating an example of a configuration of a medical information processing system including a medical information processing apparatus 100 according to a first embodiment. The medical information processing system illustrated in FIG. 1 is provided with a gene test device 200, a server 300, a terminal 50, and the medical information processing apparatus 100.

The gene test device 200 is a device for testing genes of a patient. For example, the gene test device 200 acquires information on a gene mutation related to a disease of the patient from a body fluid (blood or saliva) of the patient, as a result of testing genes of the patient, and transmits the result to the medical information processing apparatus 100.

The server 300 is provided with a gene database 310. For example, the gene database 310 stores therein information including therapeutic agents and effects of the therapeutic agents on gene mutations related to diseases in a patient group, for each disease. For example, in a case where there is a mutation of a gene a in a disease A, information including a therapeutic agent for the mutation of the gene a in the disease A and an effect of the therapeutic agent is stored in the gene database 310, and in a case where there is the mutation of the gene a in a disease B, information including a therapeutic agent for the mutation of the gene a in the disease B and an effect of the therapeutic agent is stored in the gene database 310. Regarding the effects of the therapeutic agents, contents of reports compiled by a hospital that has carried out treatment with the therapeutic agents or contents extracted from literatures on the therapeutic agents are registered in the gene database 310 as the effects of the therapeutic agents. The information stored in the gene database 310 can be read by the medical information processing apparatus 100.

The terminal 50 and the medical information processing apparatus 100 are connected to, for example, in-hospital local area network (LAN) installed in the hospital, transmit information to a predetermined device, and receive information transmitted from the predetermined device. In addition, various servers such as a hospital information system (HIS) server are connected to the in-hospital LAN. The HIS server may be connected to an external network in addition to the in-hospital LAN.

The terminal 50 is used by a doctor in the hospital. The terminal 50 includes, for example, a personal computer (PC), a tablet PC, a personal digital assistant (PDA), a mobile terminal, or the like. A viewer (software) for displaying various pieces of information on its own display is installed in the terminal 50.

The medical information processing apparatus 100 is a workstation for displaying various pieces of information on the display of the terminal 50. For example, an application (computer program) is installed in the medical information processing apparatus 100, and the application can be read by the terminal 50.

Hereinafter, the details of the medical information processing apparatus 100 according to the present embodiment will be described. FIG. 1 is a diagram illustrating an example of a configuration of the medical information processing apparatus 100 according to the present embodiment. As illustrated in FIG. 1, the medical information processing apparatus 100 includes an input interface 110, display 120, a communication interface 130, a storage circuitry 140, and a processing circuitry 150.

The input interface 110 includes a pointing device such as a mouse, a keyboard, or the like, receives inputs of various operations for the medical information processing apparatus 100 from the user, and transmits information on instructions and settings received from the user to the processing circuitry 150. Here, the user is a medical professional including a doctor.

The display 120 is a monitor referred to by the user, displays various data such as images to the user under the control of the processing circuitry 150, and displays a graphical user interface (GUI) for receiving various instructions and various settings from the user through the input interface 110. The communication interface 130 is a network interface card (NIC) or the like, and other devices communicates with each other through the communication interface 130.

The storage circuitry 140 is a semiconductor memory device such as a random-access memory (RAM) or a flash memory, or a storage device such as a hard disk or an optical disc, for example.

Furthermore, the storage circuitry 140 stores therein a patient information DB 141, a gene test result DB 142, a gene mechanism information DB 143, and a gene therapy information DB 144 as various databases (hereinafter, referred to as DBs) used by each of processing functions included in the processing circuitry 150 described below. Various DBs will be described later.

The processing circuitry 150 controls components of the medical information processing apparatus 100. For example, as illustrated in FIG. 1, the processing circuitry 150 executes an acquisition function 151, a control function 152, and a presentation function 153. Here, for example, each of the processing functions executed by the acquisition function 151, the control function 152, and the presentation function 153, which are components of the processing circuitry 150, is stored in the storage circuitry 140 in the form of a computer program that can be executed by a computer. The processing circuitry 150 is a processor that implements a function corresponding to each computer program by reading out the computer program from the storage circuitry 140 and executing each computer program. In other words, the processing circuitry 150 in a state where each computer program has been read out has each function illustrated in the processing circuitry 150 of FIG. 1. The acquisition function 151 is an example of an acquisition unit, the control function 152 is an example of a control unit, and the presentation function 153 is an example of a presentation unit.

The term “processor” used in the above description refers to, for example, a circuitry such as a central processing unit (CPU), a graphics processing unit (GPU), an application specific integrated circuit (ASIC), and programmable logic devices (for example, a simple programmable logic device (SPLD), a complex programmable logic device (CPLD), and a field programmable gate array (FPGA)). In a case where the processor is, for example, the CPU, the processor implements functions by reading out and executing computer programs stored in the storage circuitry 140. On the other hand, in a case where the processor is, for example, the ASIC, computer programs are directly incorporated in a circuitry of the processor instead of being stored in the storage circuitry 140. Each processor of the present embodiment is not limited to a case where each processor is configured as a single circuitry, and a plurality of independent circuitries may be combined to form one processor to implement the functions. Furthermore, a plurality of the components in FIG. 1 may be integrated into one processor to implement the functions.

As described above, the entire configuration of the medical information processing system including the medical information processing apparatus 100 according to the present embodiment has been described. Based on such a configuration, the medical information processing apparatus 100 proposes treatment according to the patient's request.

For example, in the hospital, as a result of testing genes of the patient, information on gene mutations related to diseases of the patient is acquired from the gene test device 200 to determine therapeutic agents suitable for the patient. In this case, for example, a doctor who operates the terminal 50 reads out information stored in the gene database 310 and determines therapeutic agents for gene mutations related to diseases of the patient as candidate agents in the order of higher therapeutic effect.

In this case, the doctor refers, for example, a screen on which a gene causing a mutation and positions where the candidate agents act are presented together with a priority of each candidate agent on a genetic map on which action mechanisms between genes are represented in in-vivo communication channel to determine a therapeutic agent to be administered to the patient. The genetic map is a correlation diagram illustrating a relationship between genes, and is an example of information representing a relationship between genes. The doctor presents the candidate agents to the patient by causing the display of the terminal 50 to display the determined candidate agents. That is, the doctor proposes the determined candidate agents to the patient as the therapeutic agents suitable for the patient.

However, in a case where the candidate agents are determined in consideration of only the therapeutic effect, the patient may not undergo treatment with the candidate agents due to various circumstances of the patient. For example, in a case where a candidate agent has a high drug price, treatment may not be carried out with the candidate agent due to economic circumstances such as the patient being unable to pay the treatment cost. In addition, in a case where side effects of a candidate agent are undesirable for the patient, the patient may abandon the continuation of treatment even though the administration of the candidate agent is started.

Therefore, the medical information processing apparatus 100 according to the present embodiment executes the following processing in order to propose treatment according to the patient's request. First, in the medical information processing apparatus 100 according to the present embodiment, the acquisition function 151 acquires gene therapy information in which candidate agents that are therapeutic agents for a gene mutation related to a disease of the patient, effects of the candidate agents, and ancillary information on the candidate agents are associated with one another. The control function 152 determines priority orders of the candidate agents based on the gene therapy information and patient information on the patient. The presentation function 153 presents the priority order in association with information representing a relationship between genes. Hereinafter, the details of the medical information processing apparatus 100 according to the first embodiment will be described.

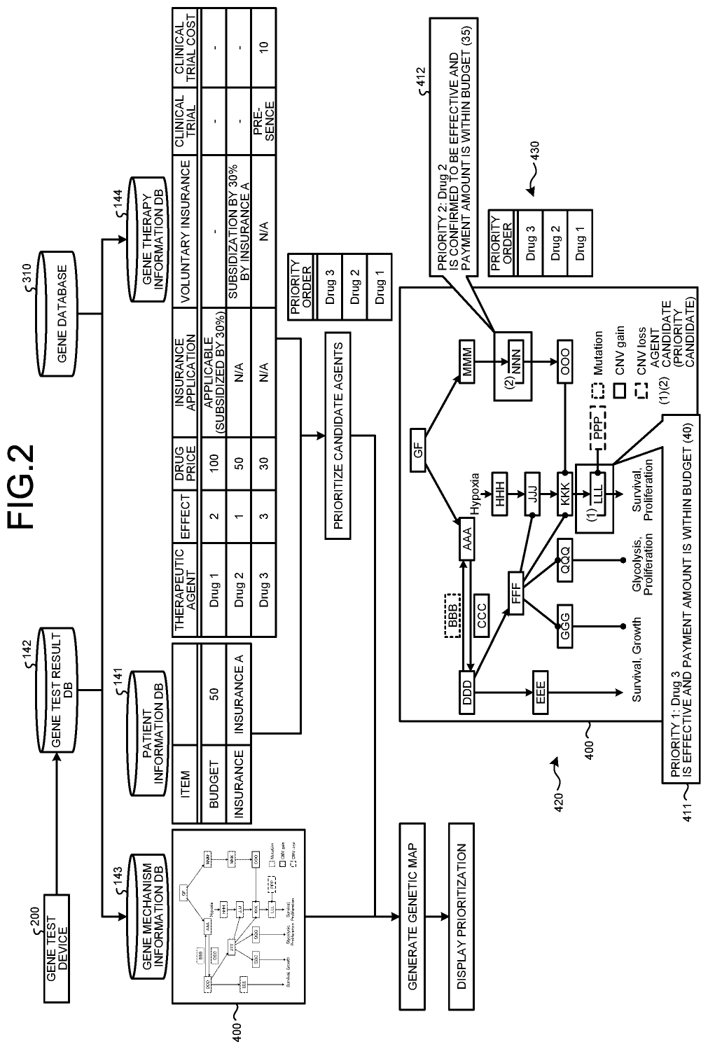

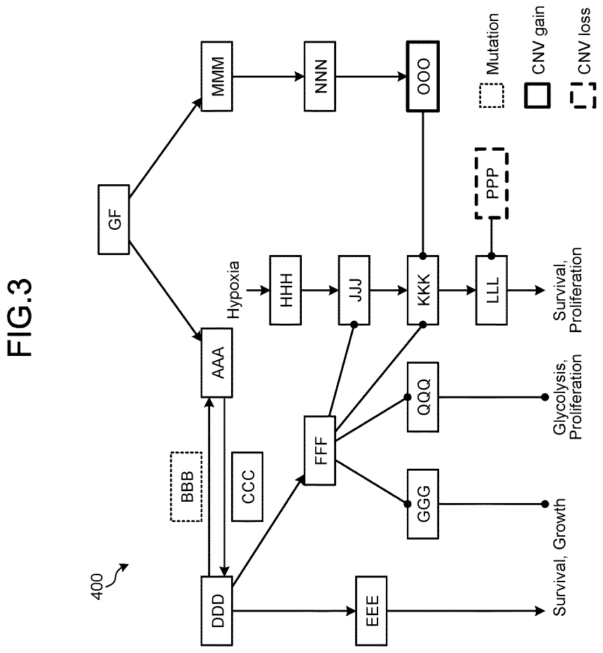

FIG. 2 is a diagram for explaining each processing function of the medical information processing apparatus 100 according to the first embodiment. FIG. 3 is a diagram illustrating an example of a genetic map according to the first embodiment. First, processing executed by the acquisition function 151 will be described with reference to FIG. 2 and FIG. 3.

The acquisition function 151 stores patient information on the patient in the patient information DB 141. For example, the doctor who operates the terminal 50 acquires the patient information from the patient by conducting an interview or a questionnaire to the patient in advance, and the acquisition function 151 stores the acquired patient information in the patient information DB 141.

The patient information stored in the patient information DB 141 includes, for example, a budget of the patient. In addition, the patient information stored in the patient information DB 141 includes information on insurance that the patient has taken out. In this case, in the example illustrated in FIG. 2, the budget of the patient is represented by “50 (ten thousand yen)”, and the insurance that the patient has taken out is represented by an “insurance A”.

For example, a test for genes of the patient is carried out with the gene test device 200. In this case, the acquisition function 151 acquires information on the gene mutation related to the disease of the patient from the gene test device 200 as a result of testing genes of the patient, and stores the acquired information in the gene test result DB 142 as the result of testing genes of the patient.

The acquisition function 151 acquires the gene therapy information in which the candidate agents that are therapeutic agents for the gene mutation related to the disease of the patient, the effects of the candidate agents, and the ancillary information on the candidate agents are associated with one another based on the information stored in the gene database 310 and the information stored in the gene test result DB 142, and stores the acquired gene therapy information in the gene therapy information DB 144. In the example illustrated in FIG. 2, in the gene therapy information stored in the gene therapy information DB 144, the candidate agents that are therapeutic agents associated with the gene mutation found in the testing result of the patient are represented by “Drug 1”, “Drug 2”, and “Drug 3”, and the effects of the candidate agents “Drug 1”, “Drug 2”, and “Drug 3” are represented by numerical values of priorities “2”, “1”, and “3”, respectively. Here, the higher the numerical values of the priorities, the higher the effects of the candidate agents.

The priority representing the effect of each agent may be registered in the gene database 310, or may be calculated by the acquisition function 151 based on the information on the effects registered in the gene database 310.

The ancillary information of the candidate agents includes, for example, drug prices of the candidate agents. In this case, in the example illustrated in FIG. 2, the drug prices of the candidate agents “Drug 1”, “Drug 2”, and “Drug 3” are represented by “100 (ten thousand yen)”, “50 (ten thousand yen)”, and “30 (ten thousand yen)”, respectively.

In addition, the ancillary information includes, for example, information on insurance to which a candidate agent is applicable. Examples of the insurance to which the candidate agent is applicable include insurance under the public medical insurance system and insurance that the patient has taken out.

In this case, in the example illustrated in FIG. 2, insurance under the public medical insurance system can be applied to the candidate agent “Drug 1”. For example, in a case where the candidate agent “Drug 1” is used as a therapeutic agent for the patient, the drug price is subsidized by 30% with respect to the drug price “100 (ten thousand yen)” of the candidate agent “Drug 1” by application of the insurance under the public medical insurance system. That is, in the case where the candidate agent “Drug 1” is used as a therapeutic agent for the patient, the payment amount of the patient is 100 (ten thousand yen)×0.3=70 (ten thousand yen) by application of the insurance under the public medical insurance system.

In addition, in the example illustrated in FIG. 2, the candidate agent “Drug 2” can be used under the “insurance A” as insurance that the patient has taken out. For example, in a case where the candidate agent “Drug 2” is used as a therapeutic agent for the patient, a drug price is subsidized by 30% with respect to the drug price “50 (ten thousand yen)” of the candidate agent “Drug 2” by application of the “insurance A”. That is, in the case where the candidate agent “Drug 2” is used as a therapeutic agent for the patient, the payment amount of the patient is 50 (ten thousand yen)×0.3=35 (ten thousand yen) by application of the insurance of “insurance A” that the patient has taken out.

Furthermore, the ancillary information includes information on whether the candidate agent is a clinical trial agent, and further includes a cost required for a clinical trial in a case where the candidate agent is the clinical trial agent, for example. Here, the clinical trial agent is a new drug developed by a pharmaceutical company, and is a drug administered to a subject such as a patient in a hospital as a clinical trial conducted to obtain approval as a “Drug” from a national institution (for example, Ministry of Health, Labour and Welfare of Japan). In addition, the clinical trial agent is also called a clinical trial drug. In the example illustrated in FIG. 2, the candidate agent “Drug 3” is a clinical trial agent, and the cost required for the clinical trial of the patient is represented by “10 (ten thousand yen)”. In this case, in the case where the candidate agent “Drug 3” is used as a therapeutic agent for the patient, the payment amount of the patient is 30 (ten thousand yen)+10 (ten thousand yen)=40 (ten thousand yen) in consideration of the clinical trial cost “10 (ten thousand yen)” of the patient.

In addition, the acquisition function 151 acquires a genetic map 400 that is a correlation diagram illustrating a relationship between genes based on the information stored in the gene database 310 and the information stored in the gene test result DB 142, and stores the acquired genetic map 400 in the gene mechanism information DB 143 as gene mechanism information. The genetic map 400 is an example of information representing the relationship between genes. The genetic map 400 illustrated in FIG. 2 is enlarged and illustrated in FIG. 3.

In the genetic map 400 illustrated in FIG. 3, “GF”, “DDD”, “AAA”, “BBB”, and the like illustrate biomolecules such as proteins produced by gene expression.

It is illustrated in FIG. 3 that expression of the biomolecule “GF” promotes expression of a biomolecule “MMM”, the biomolecule “MMM” promotes expression of a biomolecule “NNN”, and the biomolecule “NNN” promotes expression of a biomolecule “OOO”.

In addition, it is illustrated in FIG. 3 that the expression of the biomolecule “GF” promotes expression of the biomolecule “AAA” as another pathway generated by the biomolecule “GF”. In addition, it is illustrated in FIG. 3 that the biomolecule “AAA” changes into the biomolecule “DDD” due to a biomolecule “CCC”, and the biomolecule “DDD” changes into the biomolecule “AAA” due to the biomolecule “BBB”.

In addition, it is illustrated in FIG. 3 that the biomolecule “DDD” promotes expression of a biomolecule “EEE”, and as a result, survival of cells is promoted. In addition, it is illustrated in FIG. 3 that a biomolecule “FFF” suppresses expression of a biomolecule “GGG”, and as a result, growth of cells is suppressed. In addition, it is illustrated in FIG. 3 that the biomolecule “FFF” suppresses expression of a biomolecule “QQQ”, and as a result, glycolysis is suppressed, and proliferation of cells is suppressed.

In addition, it is illustrated in FIG. 3 that in a state where cells are in hypoxia, expression of a biomolecule “HHH” is promoted, the biomolecule “HHH” promotes expression of a biomolecule “JJJ”, the biomolecule “JJJ” promotes expression of a biomolecule “KKK”, the biomolecule “KKK” promotes expression of a biomolecule “LLL”, and as a result, survival and proliferation of cells are promoted. In addition, it is illustrated in FIG. 3 that the biomolecule “FFF” suppresses the expression of biomolecule “JJJ” and biomolecule “KKK”, and a biomolecule “PPP” suppresses the expression of the biomolecule “LLL”.

In the above description, although the line whose end point is an arrow has been described as promoting expression, the line may also be used as promoting function. In addition, although the line whose end point is a circular arrow has been described as suppressing expression, the line may also be used as suppressing function.

It is illustrated in FIG. 3 that a gene encoding the biomolecule “BBB” is mutated according to the gene test result of the patient. In addition, it is illustrated in FIG. 3 that the copy number of the gene encoding the biomolecule “OOO” is increasing according to the gene test result of the patient (“copy number variation (CNV) gain”). In addition, it is illustrated in FIG. 3 that the copy number of the gene encoding the biomolecule “PPP” is decreasing according to the gene test result of the patient (“CNV loss”).

The above-described candidate agents “Drug 1”, “Drug 2”, and “Drug 3” are, for example, therapeutic agents whose therapeutic effects have been confirmed for a disease caused by the mutation of the gene encoding the biomolecule “BBB”.

Next, processing executed by the control function 152 will be described with reference to FIG. 2. The control function 152 determines priority orders of the candidate agents based on the gene therapy information stored in the gene therapy information DB 144 and the patient information stored in the patient information DB 141.

In the example illustrated in FIG. 2, in a case where the effects of the candidate agents “Drug 1”, “Drug 2”, and “Drug 3” correspond to numerical values of priorities “2”, “1”, and “3”, respectively, and the candidate agents “Drug 1”, “Drug 2”, and “Drug 3” are used as therapeutic agents for the patient, payment amounts of the patient are 70 (ten thousand yen), 35 (ten thousand yen), and 40 (ten thousand yen), respectively. In this case, the control function 152 determines priority orders of the candidate agents based on the effects of the candidate agents “Drug 1”, “Drug 2”, and “Drug 3” and the payment amounts (costs) borne by the patient.

For example, since the candidate agent “Drug 3” is the most effective of the candidate agents “Drug 1”, “Drug 2”, and “Drug 3”, and the payment amount (400,000 yen) is within the budget of the patient, the candidate agent “Drug 3” has the first priority order. In addition, since the candidate agent “Drug 2” is confirmed to be effective and the payment amount (350,000 yen) is within the budget of the patient, the candidate agent “Drug 2” has the second priority order. Although the candidate agent “Drug 1” is the next most effective after “Drug 3”, the payment amount is 700,000 yen, which is not within the budget of the patient, so that the candidate agent “Drug 1” has the third priority order.

The control function 152 generates support information 420 in which the determined priority orders are associated with the genetic map 400 stored in the gene mechanism information DB 143.

Next, processing executed by the presentation function 153 will be described with reference to FIG. 2. The presentation function 153 presents the doctor and the patient with the support information 420 and an order table 430 representing the priority orders of the candidate agents “Drug 1”, “Drug 2”, and “Drug 3” by displaying the support information 420 and the order table 430 on the display of the terminal 50 of the doctor.

For example, in a case where the support information 420 is displayed on the display of the terminal 50, the presentation function 153 presents the priority order at a position where the candidate agents act in the genetic map 400. In this case, in the example illustrated in FIG. 2, “(1)” indicating the first priority order is displayed on the biomolecule “LLL” as a position where the candidate agent “Drug 3” acts in the genetic map 400, and “(2)” indicating the second priority order is displayed on the biomolecule “NNN” as a position where the candidate agent “Drug 2” acts in the genetic map 400.

In addition, in a case where the support information 420 is displayed on the display of the terminal 50, the presentation function 153 presents the reasons for determining the priority orders of the candidate agents. Specifically, the presentation function 153 presents the priority orders of the candidate agents and the reasons by displaying comments 411 and 412 in the genetic map 400 on the display of the terminal 50. In this case, in the example illustrated in FIG. 2, the comment 411 indicates the biomolecule “LLL” as the position where the candidate agent “Drug 3” acts in the genetic map 400, and “Priority 1: Drug 3 is highly effective and the payment amount is within the budget (40)” is displayed as the comment 411. In addition, the comment 412 indicates the biomolecule “NNN” as the position where the candidate agent “Drug 2” acts in the genetic map 400, and “Priority 2: Drug 2 is confirmed to be effective and the payment amount is within the budget (35)” is displayed as the comment 412.

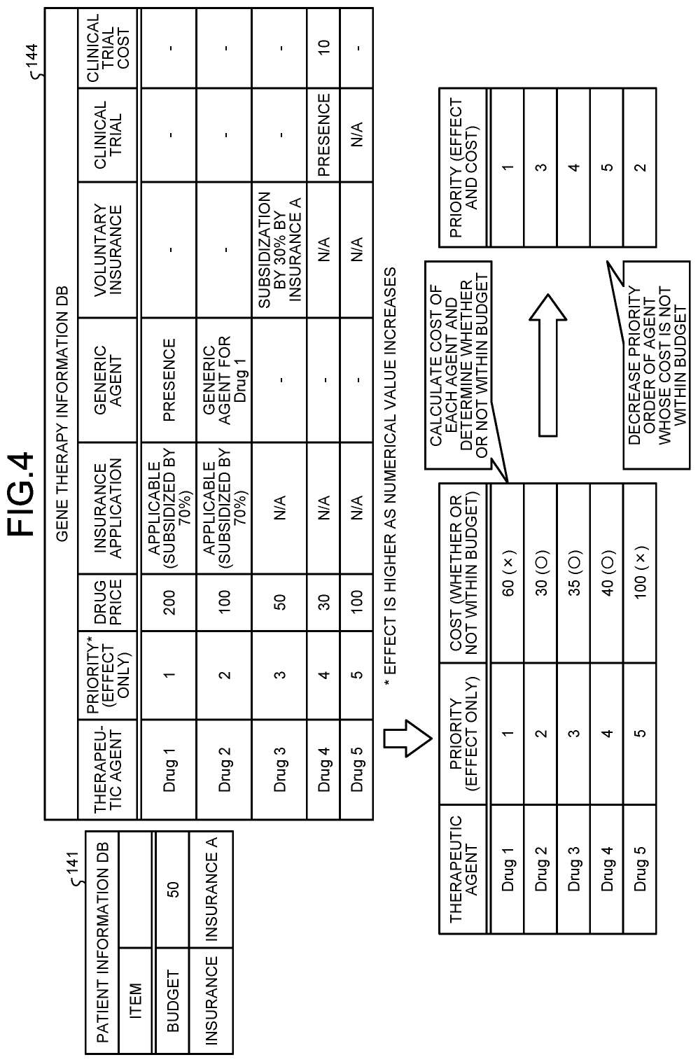

Here, a specific example different from the processing of FIG. 2 will be described with reference to FIG. 4. FIG. 4 is a diagram illustrating a processing example different from the processing example illustrated in FIG. 2.

As described above, the patient information stored in the patient information DB 141 includes the budget of the patient and information on insurance that the patient has taken out. In this case, in the example illustrated in FIG. 4, the budget of the patient is represented by “50 (ten thousand yen)”, and the insurance that the patient has taken out is represented by an “insurance A”.

As described above, the gene therapy information stored in the gene therapy information DB 144 is information in which the candidate agents that are therapeutic agents for a gene mutation related to a disease of the patient, the effects of the candidate agents, and the ancillary information of the candidate agents are associated with one another. In this case, in the example illustrated in FIG. 4, the candidate agents that are therapeutic agents are represented by “Drug 1”, “Drug 2”, “Drug 3”, “Drug 4”, and “Drug 5”, and the effects of the candidate agents “Drug 1”, “Drug 2”, “Drug 3”, “Drug 4”, and “Drug 5” are represented by numerical values of priorities “1”, “2”, “3”, “4”, and “5”, respectively. Here, the higher the numerical values of the priorities, the higher the effects of the candidate agents.

In the gene therapy information, as described above, the ancillary information includes drug prices of the candidate agents, information on insurance to which candidate agents are applicable, and information on clinical trials to which the candidate agents are applicable. Furthermore, the ancillary information includes, for example, information on whether a generic agent is included in the candidate agents. In this case, in the example illustrated in FIG. 4, the drug prices of the candidate agents “Drug 1”, “Drug 2”, “Drug 3”, “Drug 4”, and “Drug 5” are represented by “200 (ten thousand yen)”, “100 (ten thousand yen)”, “50 (ten thousand yen)”, “30 (ten thousand yen)”, and “100 (ten thousand yen)”, respectively. In addition, there is a generic agent for the candidate agent “Drug 1”. For example, the candidate agent “Drug 2” is a generic agent for the candidate agent “Drug 1”. For example, the effect of the candidate agent “Drug 2” is equal to or slightly higher than the effect of the candidate agent “Drug 1”. In the example illustrated in FIG. 4, the effect of the candidate agent “Drug 2” is slightly higher than the effect of the candidate agent “Drug 1”.

In addition, in the example illustrated in FIG. 4, in a case where the candidate agents “Drug 1” and “Drug 2” are used as therapeutic agents for the patient, drug prices are subsidized by 70° with respect to the drug prices of the candidate agents “Drug 1” and “Drug 2” by application of the insurance under the public medical insurance system. In a case where the candidate agent “Drug 3” is used as a therapeutic agent for the patient, a drug price is subsidized by 30% with respect to the drug price of the candidate agent “Drug 3” by application of the “insurance A”. In addition, the candidate agent “Drug 4” is a clinical trial agent, and a cost required for the clinical trial of the patient is represented by “10 (ten thousand yen)”. In a case where the clinical trial is conducted at a different hospital from the patient's hospital, the clinical trial cost includes expenses such as costs for transportation to the hospital.

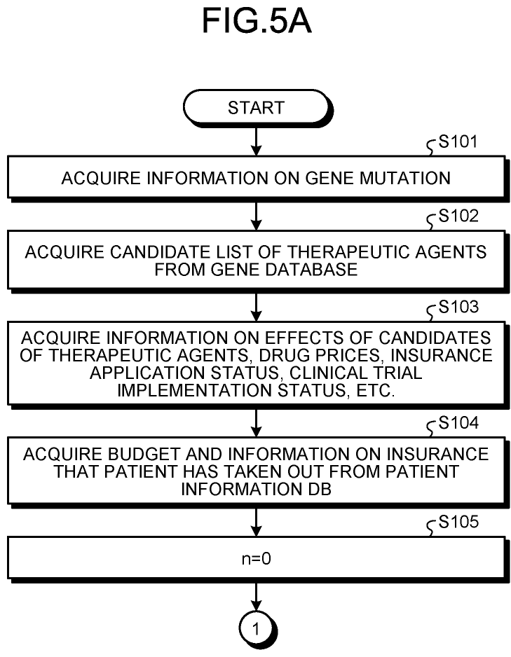

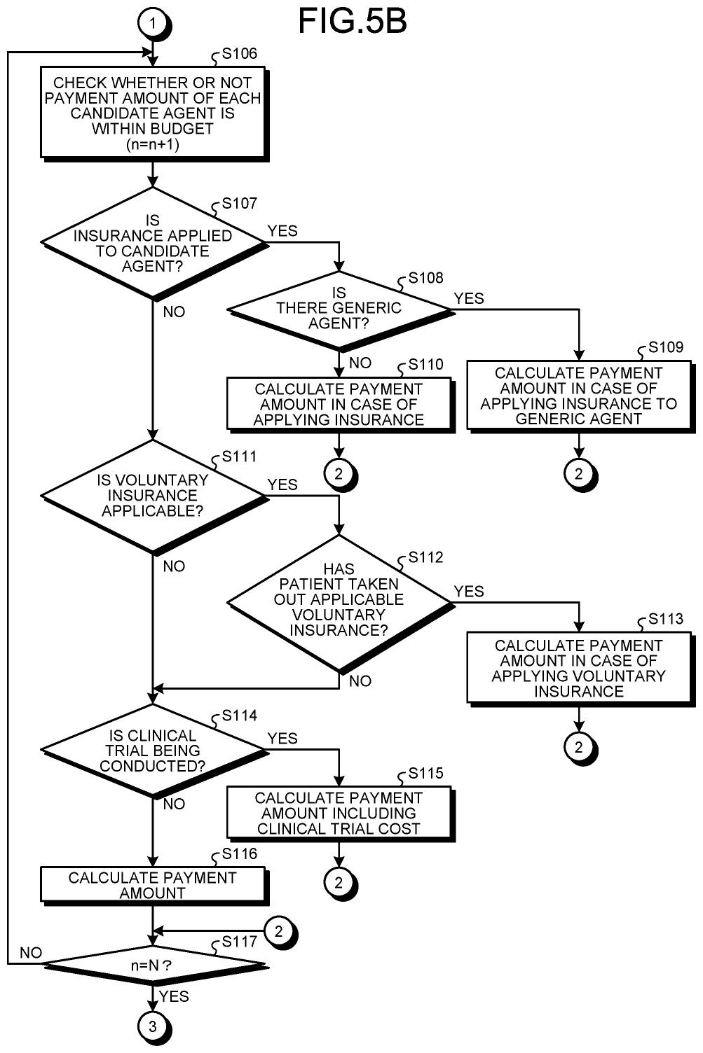

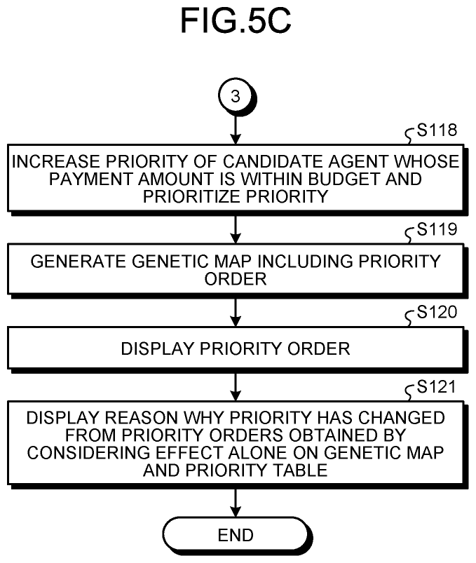

Processing procedures will be described with reference to the example illustrated in FIG. 4. FIG. 5A to FIG. 5C are flowcharts illustrating processing procedures executed by the medical information processing apparatus 100 according to the first embodiment.

First, at step S101 of FIG. 5A, the acquisition function 151 acquires information on a gene mutation related to the disease of the patient. Specifically, the acquisition function 151 acquires information on the gene mutation related to the disease of the patient from the gene test device 200 as a result of testing genes of the patient, and stores the acquired information in the gene test result DB 142 as the result of testing genes of the patient.

Next, at step S102 of FIG. 5A, the acquisition function 151 acquires a candidate list of therapeutic agents from the information stored in the gene database 310. Specifically, the acquisition function 151 acquires candidate agents “Drug 1”, “Drug 2”, “Drug 3”, “Drug 4”, and “Drug 5”, which are therapeutic agents for the gene mutation related to the disease of the patient, based on the information stored in the gene database 310 and the information stored in the gene test result DB 142.

Next, at step S103 of FIG. 5A, the acquisition function 151 acquires the effects of the candidate agents “Drug 1”, “Drug 2”, “Drug 3”, “Drug 4”, and “Drug 5” and information such as drug prices, information on insurance to which the candidate agents are applicable, and information on clinical trials to which the candidate agents are applicable as the ancillary information of the candidate agents, based on the information stored in the gene database 310 and the information stored in the gene test result DB 142. The acquisition function 151 then stores gene therapy information in which the candidate agents “Drug 1”, “Drug 2”, “Drug 3”, “Drug 4”, and “Drug 5”, effects of the candidate agents, and ancillary information of the candidate agents are associated with one another in the gene therapy information DB 144.

Next, at step S104 of FIG. 5A, the acquisition function 151 acquires the budget “50 (ten thousand yen)” of the patient and the “insurance A” that is the information on the insurance that the patient has taken out from the patient information stored in the patient information DB 141.

Here, at step S105 of FIG. 5A, since the number of candidate agents is 5, the control function 152 sets N=5, and assuming that the candidate agents “Drug 1”, “Drug 2”, “Drug 3”, “Drug 4”, and “Drug 5” are set to n=1, n=2, n=3, n=4, and n=5, respectively, the count n is currently set to “0 (n=0)”.

At step S106 of FIG. 5B, the control function 152 checks whether the payment amount of each candidate agent is within the budget. In this case, the control function 152 increments the count n by 1 (n=n+1) and executes processing of calculating the payment amount in a case where the candidate agent “Drug 1” is used as a therapeutic agent for the patient. Specifically, in the case where the candidate agent “Drug 1” is used, in the example illustrated in FIG. 4, the insurance under the public medical insurance system can be applied to the candidate agent “Drug 1” (Yes at step S107). In addition, there is a generic agent for the candidate agent “Drug 1” (Yes at step S108). In this case, the control function 152 calculates 100 (ten thousand yen)×0.7=30 (ten thousand yen) as the payment amount in a case of applying the insurance under the public medical insurance system to the candidate agent “Drug 2” that is the generic agent for the candidate agent “Drug 1” (step S109). Here, although not illustrated in the processing flow of FIG. 5B, the control function 152 also calculates the payment amount in a case of applying the insurance under the public medical insurance system to the candidate agent “Drug 1”. In this case, the control function 152 calculates 200 (ten thousand yen)×0.7=60 (ten thousand yen) as the payment amount in the case of applying the insurance under the public medical insurance system to the candidate agent “Drug 1”.

In a case where the insurance under the public medical insurance system can be applied to the candidate agent (Yes at step S107), but there is no generic agent for the candidate agent (No at step S108), the control function 152 calculates the payment amount in a case of applying the insurance under the public medical insurance system to the candidate agent (step S110).

Here, since the count n is currently “1 (n=1)” and not “5 (N=5)” (No at step S117), the processing executed by the control function 152 returns to step S106.

Next, the control function 152 increments the count n by (step S106) and executes processing of calculating the payment amount in a case where the candidate agent “Drug 3” is used as a therapeutic agent for the patient. Specifically, in the case where the candidate agent “Drug 3” is used, in the example illustrated in FIG. 4, although the insurance under the public medical insurance system cannot be applied to the candidate agent “Drug 3” (No at step S107), voluntary insurance can be applied (Yes at step S111). Here, the patient has taken out the “insurance A” that is applicable as voluntary insurance (Yes at step S112). In this case, the control function 152 calculates 50 (ten thousand yen)×0.3=35 (ten thousand yen) as the payment amount in a case of applying the “insurance A” that the patient has taken out to the candidate agent “Drug 3” (step S113). Since the count n is currently “3 (n=3)” and not “5 (N=5)” (No at step S117), the processing executed by the control function 152 returns to step S106.

Next, the control function 152 increments the count n by (step S106) and executes processing of calculating the payment amount in a case where the candidate agent “Drug 4” is used as a therapeutic agent for the patient. Specifically, in the case where the candidate agent “Drug 4” is used, in the example illustrated in FIG. 4, the insurance under the public medical insurance system cannot be applied to the candidate agent “Drug 4” (No at step S107), and voluntary insurance cannot be applied (No at step S111). Alternatively, the voluntary insurance can be applied (Yes at step S111), but the patient has no voluntary insurance (No at step S112). Here, the candidate agent “Drug 4” is a clinical trial agent, and a cost required for the clinical trial of the patient is represented by “10 (ten thousand yen)” (Yes at step S114). In this case, the control function 152 calculates 30 (ten thousand yen)+10 (ten thousand yen)=40 (ten thousand yen) as the payment amount in a case where the clinical trial cost (100,000 yen) of the patient is added for the candidate agent “Drug 4” (step S115). Since the count n is currently “4 (n=4)” and not “5 (N=5)” (No at step S117), the processing executed by the control function 152 returns to step S106.

Next, the control function 152 increments the count n by (step S106) and executes processing of calculating the payment amount in a case where the candidate agent “Drug 5” is used as a therapeutic agent for the patient. Specifically, in the case where the candidate agent “Drug 5” is used, in the example illustrated in FIG. 4, the insurance under the public medical insurance system cannot be applied to the candidate agent “Drug 5” (No at step S107), and voluntary insurance cannot be applied (No at step S111). In addition, it is assumed that no clinical trial is being conducted on the patient (No at step S114). In this case, the control function 152 calculates the payment amount of the patient as 100 (ten thousand yen) for the candidate agent “Drug 5” (step S116). Since the count n is currently “5 (N=5)” (Yes at step S117), the processing executed by the control function 152 ends.

The processing flow illustrated in FIG. 5B is an example, and the order and contents in the processing flow executed by the control function 152 are not limited thereto. For example, in the processing flow illustrated in FIG. 5B, in a case where there is a generic agent for the candidate agent, the payment amount in a case of applying the insurance under the public medical insurance system to the generic agent is calculated, but in a case where there is a generic agent for the candidate agent, the payment amount in a case of applying the insurance under the public medical insurance system to both the candidate agent and the generic agent for the candidate agent may be calculated. In addition, the same percentage of the drug price subsidized by application of the insurance under the public medical insurance system is applied to the candidate agent and the generic agent for the candidate agent, but the different percentage may be applied.

Next, at step S118 of FIG. 5C, the control function 152 increases a priority of a candidate agent whose payment amount is within the budget of the patient and prioritizes the priority. In the example illustrated in FIG. 4, in a case where the effects of the candidate agents “Drug 1”, “Drug 2”, “Drug 3”, “Drug 4”, and “Drug 5” correspond to numerical values of priorities “1”, “2”, “3”, “4”, and “5”, respectively, and the candidate agents “Drug 1”, “Drug 2”, “Drug 3”, “Drug 4”, and “Drug 5” are used as therapeutic agents for the patient, payment amounts of the patient are 60 (ten thousand yen), 30 (ten thousand yen), 35 (ten thousand yen), 40 (ten thousand yen), and 100 (ten thousand yen), respectively. In this case, the control function 152 determines priority orders of the candidate agents based on the effects of the candidate agents “Drug 1”, “Drug 2”, “Drug 3”, “Drug 4”, and “Drug 5” and the payment amounts (costs) of the patient.

In this case, in the example illustrated in FIG. 4, since the candidate agent “Drug 4” is the next most effective after the candidate agent “Drug 5”, and the payment amount (400,000 yen) is within the budget of the patient, the priority of the candidate agent “Drug 4” is increased from “4” to “5”, and the candidate agent “Drug 4” has the first priority order. In addition, since the candidate agent “Drug 3” is the next most effective after the candidate agent “Drug 4”, and the payment amount (350,000 yen) is within the budget of the patient, the priority of the candidate agent “Drug 3” is increased from “3” to “4”, and the candidate agent “Drug 3” has the second priority order. In addition, since the candidate agent “Drug 2” is the next most effective after the candidate agent “Drug 3”, and the payment amount (300,000 yen) is within the budget of the patient, the priority of the candidate agent “Drug 2” is increased from “1” to “3”, and the candidate agent “Drug 2” has the third priority order. In addition, although the candidate agent “Drug 5” is the most effective, the payment amount is 1,000,000 yen, which is not within the budget of the patient. Therefore, the priority of the candidate agent “Drug 5” is decreased from “5” to “2”, and the candidate agent “Drug 5” has the fourth priority order. In addition, although the candidate agent “Drug 1” is more effective than the candidate agent “Drug 2”, the payment amount is 600,000 yen, which is not within the budget of the patient. Therefore, the priority of the candidate agent “Drug 1” remains at “1”, and the candidate agent “Drug 1” has the fifth priority order.

Next, at step S119 of FIG. 5C, the control function 152 generates the genetic map 400 including the determined priority orders. Specifically, the control function 152 generates the support information 420 in which the determined priority orders are associated with the genetic map 400 stored in the gene mechanism information DB 143.

Next, at step S120 of FIG. 5C, the presentation function 153 presents to the doctor and the patient the priority orders by displaying the priority orders on the display of the terminal 50 of the doctor. Specifically, the presentation function 153 presents to the doctor and the patient the support information 420 and the order table 430 representing the priority orders of the candidate agents “Drug 1”, “Drug 2”, “Drug 3”, “Drug 4”, and “Drug 5”.

Next, at step S121 of FIG. 5C, the presentation function 153 displays a reason why the priority has changed from the priority orders obtained by considering the effect alone on the support information 420 and the order table 430 that is a priority table. Specifically, the presentation function 153 presents the priority orders of the candidate agents and the reasons by displaying first to third comments in the genetic map 400 on the display of the terminal 50. For example, the first comment indicates a position where the candidate agent “Drug 4” acts in the genetic map 400, and “Priority 1: Drug 4 is effective and the payment amount is within the budget (40)” is displayed as the comment. In addition, the second comment indicates a position where the candidate agent “Drug 3” acts in the genetic map 400, and “Priority 2: Drug 3 is the next most effective after “Drug 4” and the payment amount is within the budget (35)” is displayed as the comment. In addition, the third comment indicates a position where the candidate agent “Drug 2” acts in the genetic map 400, and “Priority 3: Drug 2 is the next most effective after “Drug 3” and the payment amount is within the budget (30)” is displayed as the comment. Here, as the third comment, it is further displayed that the candidate agent “Drug 2” is a generic agent for the candidate agent “Drug 1”.

With the above description, in the medical information processing apparatus 100 according to the first embodiment, the acquisition function 151 acquires the gene therapy information in which the candidate agents that are therapeutic agents for the gene mutation related to the disease of the patient, the effects of the candidate agents, and the ancillary information on the candidate agents are associated with one another. The control function 152 then determines the priority orders of the candidate agents based on the gene therapy information and the patient information on the patient, and the presentation function 153 presents the priority orders in association with the information representing the relationship between genes. As described above, the medical information processing apparatus 100 according to the first embodiment presents the priority orders of the candidate agents in consideration of not only the therapeutic effects, but also the budget of the patient and the information on the insurance that the patient has taken out as the patient information stored in the patient information DB 141, so that the patient can select the candidate agent according to the patient's desire.

Therefore, by using the medical information processing apparatus 100 according to the first embodiment, treatment can be carried out with the candidate agent according to the patient's desire, so that it is possible to propose the treatment according to the patient's request.

### Second Embodiment

In the first embodiment, the case where the priority orders of the candidate agents are determined according to the budget of the patient has been described, but the embodiment is not limited thereto. In a second embodiment, a case where the priority orders of the candidate agents are determined according to a level of living desired by the patient will be described. Specifically, in the second embodiment, the patient information stored in the patient information DB 141 is information on a level of living desired by the patient. For example, the patient may accept restrictions on physically intense activities by treatment being carried out, but desires to be able to walk and perform light work, and it is assumed that a side effect of a first candidate agent is that “it is possible to walk but not possible to work”, and a side effect of a second candidate agent is that “it is possible to do the same daily life as before the onset of illness without restriction”. In this case, the control function 152 determines priority orders of the candidate agents by prioritizing the priorities such that the priority of the second candidate agent is set to be higher than the priority of the first candidate agent, and the presentation function 153 presents the priority orders in association with the information representing the relationship between genes.

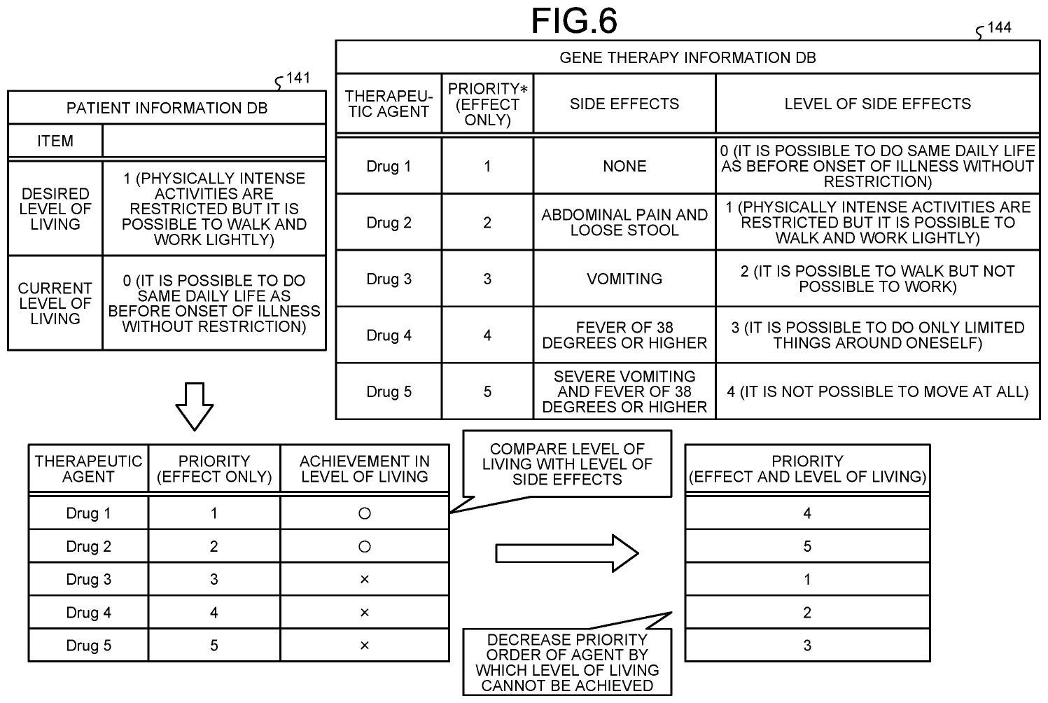

FIG. 6 is a diagram illustrating processing executed by the medical information processing apparatus 100 according to the second embodiment.

For example, the patient information stored in the patient information DB 141 includes information on the level of living desired by the patient, as described above. In this case, in the example illustrated in FIG. 6, in a case where the patient currently has a life in which “it is possible to do the same daily life as before the onset of illness without restriction”, the current level of living of the patient is represented by “0”, and in a case where the patient desires a life in which “physically intense activities are restricted but it is possible to walk and work lightly” by the treatment being carried out, the level of living desired by the patient is represented by “1”.

For example, the gene therapy information stored in the gene therapy information DB 144 is information in which candidate agents that are therapeutic agents for a gene mutation related to a disease of the patient, effects of the candidate agents, and ancillary information of the candidate agents are associated with one another. In this case, in the example illustrated in FIG. 6, the candidate agents that are therapeutic agents are represented by “Drug 1”, “Drug 2”, “Drug 3”, “Drug 4”, and “Drug 5”, and the effects of the candidate agents “Drug 1”, “Drug 2”, “Drug 3”, “Drug 4”, and “Drug 5” are represented by numerical values of priorities “1”, “2”, “3”, “4”, and “5”, respectively. Here, the higher the numerical values of the priorities, the higher the effects of the candidate agents.

In the gene therapy information, as described above, the ancillary information includes information on side effects of the candidate agents. In this case, in the example illustrated in FIG. 6, the side effects of the candidate agents “Drug 1”, “Drug 2”, “Drug 3”, “Drug 4”, and “Drug 5” are represented by “none”, “abdominal pain and loose stool”, “vomiting”, “fever of 38 degrees or higher”, and “severe vomiting and fever of 38 degrees or higher”, respectively.

In addition, in the example illustrated in FIG. 6, in a case where a side effect of the candidate agent “Drug 1” is “none”, the life of the patient is “it is possible to do the same daily life as before the onset of illness without restriction”. Therefore, a level of side effects is represented by “0”. In addition, in a case where a side effect of the candidate agent “Drug 2” is “abdominal pain and loose stool”, the life of the patient is “physically intense activities are restricted but it is possible to walk and work lightly”. Therefore, the level of side effects is represented by “1”. In addition, in a case where a side effect of the candidate agent “Drug 3” is “vomiting”, the life of the patient is “it is possible to walk but not possible to work”. Therefore, the level of side effects is represented by “2”. In addition, in a case where a side effect of the candidate agent “Drug 4” is “fever of 38 degrees or higher”, the life of the patient is “it is possible to do only limited things around oneself”. Therefore, the level of side effects is represented by “3”. In addition, in a case where a side effect of the candidate agent “Drug 5” is “fever of 38 degrees or higher”, the life of the patient is “it is not possible to move at all”. Therefore, the level of side effects is represented by “4”.

In this case, the control function 152 determines the priority orders of the candidate agents based on the gene therapy information stored in the gene therapy information DB 144 and the patient information stored in the patient information DB 141. Specifically, the control function 152 determines the priority orders of the candidate agents based on the side effects and the level of side effects of the candidate agents “Drug 1”, “Drug 2”, “Drug 3”, “Drug 4”, and “Drug 5” and the level of living desired by the patient.

For example, the candidate agents “Drug 3”, “Drug 4”, and “Drug 5” are highly effective, but the level of side effects of the candidate agents is the level of living “1” desired by the patient, and the patient cannot have the life in which “physically intense activities are restricted but it is possible to walk and work lightly”. The candidate agents “Drug 1” and “Drug 2” are less effective, but the level of side effects of the candidate agents is the level of living “1” desired by the patient, and the patient can have the life in which “physically intense activities are restricted but it is possible to walk and work lightly”.

Here, in a case where any of the candidate agent “Drug 1” or “Drug 2” is used as a therapeutic agent for the patient, since the candidate agent “Drug 2” is more effective than the candidate agent “Drug 1”, the candidate agent “Drug 2” has the first priority order, and the candidate agent “Drug 1” has the second priority order. In addition, in a case where any of the candidate agent “Drug 3”, “Drug 4”, or “Drug 5” is used as a therapeutic agent for the patient, in the order of therapeutic effects, the candidate agent “Drug 5” has the third priority order, the candidate agent “Drug 4” has the fourth priority order, and the candidate agent “Drug 3” has the fifth priority order.

In this case, the control function 152 generates the support information 420 in which the determined priority orders are associated with the genetic map 400 stored in the gene mechanism information DB 143. The presentation function 153 then presents to the doctor and the patient the support information 420 and the order table 430 representing the priority orders of the candidate agents “Drug 1”, “Drug 2”, “Drug 3”, “Drug 4”, and “Drug 5” by displaying the support information 420 and the order table 430 on the display of the terminal 50 of the doctor.

For example, in a case where the support information 420 is displayed on the display of the terminal 50, the presentation function 153 presents the priority orders at positions where the candidate agents act in the genetic map 400. In this case, in the example illustrated in FIG. 6, “(1)” indicating the first priority order is displayed on a position where the candidate agent “Drug 2” acts in the genetic map 400, and “(2)” indicating the second priority order is displayed on a position where the candidate agent “Drug 1” acts in the genetic map 400.

In addition, in a case where the support information 420 is displayed on the display of the terminal 50, the presentation function 153 presents the reasons for determining the priority orders of the candidate agents. Specifically, the presentation function 153 presents the priority orders of the candidate agents and the reasons by displaying first and second comments in the genetic map 400 on the display of the terminal 50. In this case, for the example illustrated in FIG. 6, the first comment indicates the position where the candidate agent “Drug 2” acts in the genetic map 400, and “Priority 1: Drug 2 has a high priority (effect and level of living)” is displayed as the first comment. In addition, the second comment indicates the position where the candidate agent “Drug 1” acts in the genetic map 400, and “Priority 2: Drug 1 has the next high priority (effect and level of living)” is displayed as the second comment.

As described above, the medical information processing apparatus 100 according to the second embodiment presents the priority orders of the candidate agents in consideration of not only the therapeutic effects, but also the information on the level of living desired by the patient as the patient information, so that the patient can select the candidate agent according to the patient's desire. Therefore, by using the medical information processing apparatus 100 according to the second embodiment, treatment can be carried out with the candidate agent according to the patient's desire, so that it is possible to propose the treatment according to the patient's request.

In the medical information processing apparatus 100 according to the second embodiment, an example of the patient information stored in the patient information DB 141 includes the level of living desired by the patient, and an example of the level of living includes the side effects of the candidate agents. However, the present embodiment is not limited thereto, and the level of living desired by the patient may be an administration method of a candidate agent. In this case, in the gene therapy information stored in the gene therapy information DB 144, the ancillary information includes information on the administration method of the candidate agent. For example, it is assumed that the patient desires to be able to have the same daily life as before the onset of illness, and an administration method of the first candidate agent is “prescription once every two weeks at an outpatient clinic”, and an administration method of the second candidate is “continuous infusion for one week after hospitalization”. In this case, the control function 152 determines the priority orders of the candidate agents by prioritizing the priorities such that the priority of the first candidate agent is set to be higher than the priority of the second candidate agent, and the presentation function 153 presents the priority orders in association with the information representing the relationship between genes.

In addition, in the medical information processing apparatus 100 according to the second embodiment, the example of the patient information stored in the patient information DB 141 includes the level of living desired by the patient, but the patient information may include the level of living desired by the patient in addition to the budget of the patient and the information on the insurance that the patient has taken out. For example, it is assumed that the patient desires that physically intense activities may be restricted by treatment being carried out as long as the payment amount is within the budget, and although the payment amount of the first candidate agent is within the budget, the side effect of the first candidate agent is “it is possible to walk but not possible to work”, and although the side effect of the second candidate agent is “it is possible to do the same daily life as before the onset of illness without restriction”, the payment amount of the second candidate agent is not within the budget. In this case, the control function 152 determines the priority orders of the candidate agents by prioritizing the priorities such that the priority of the first candidate agent is set to be higher than the priority of the second candidate agent, and the presentation function 153 presents the priority orders in association with the information representing the relationship between genes.

In addition, in the medical information processing apparatus 100 according to the first and second embodiments, the patient information stored in the patient information DB 141 may further include a medication history of the patient. In this case, in the gene therapy information stored in the gene therapy information DB 144, the ancillary information includes information on components included in the candidate agents. For example, in a case where there is information indicating that a candidate agent has accumulative toxicity of platinum, the acquisition function 151 acquires the accumulated amount of platinum from the medication history of the patient. Information such as the medication history of the patient is acquired from the HIS server as electronic medical record information, for example. The control function 152 decreases the priority order of the candidate agent in a case where an amount to be accumulated by the administration of the candidate agent exceeds an amount that can be ingested in the lifetime.

### Third Embodiment

In the medical information processing apparatus 100 according to a third embodiment, the control function 152 receives a change in the patient information from the terminal 50 of the doctor, and redetermines the priority orders of the candidate agents based on information after the change is received. For example, the acquisition function 151 updates the patient information in a case where the budget of the patient is changed, a case where the patient newly contracts voluntary insurance, or a case where the level of living desired by the patient is changed, as the patient information stored in the patient information DB 141. In addition, in the gene therapy information stored in the gene therapy information DB 144, in a case where there is a change in the drug price of the agent, the availability of the insurance, or the clinical trial implementation status as the ancillary information, the acquisition function 151 updates the ancillary information.

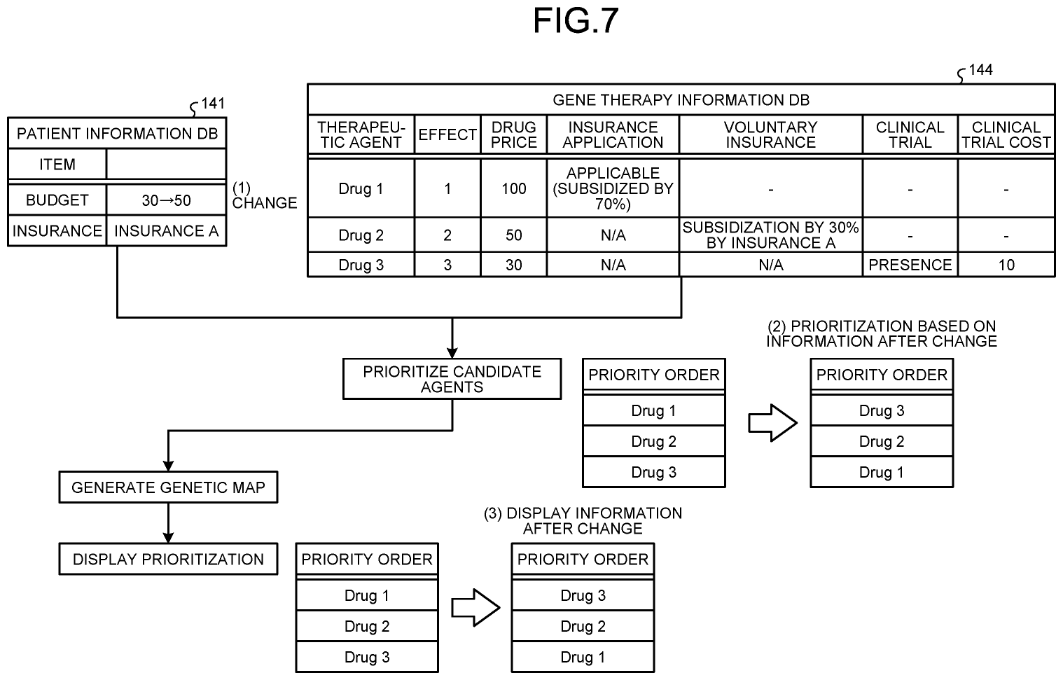

FIG. 7 is a diagram illustrating processing executed by the medical information processing apparatus 100 according to the third embodiment.

For example, the patient information stored in the patient information DB 141 includes the budget of the patient and information on insurance that the patient has taken out. In this case, in the example illustrated in FIG. 7, the budget of the patient is represented by “30 (ten thousand yen)”, and the insurance that the patient has taken out is represented by the “insurance A”.

For example, the gene therapy information stored in the gene therapy information DB 144 is information in which the candidate agents that are therapeutic agents for a gene mutation related to a disease of the patient, the effects of the candidate agents, and the ancillary information of the candidate agents are associated with one another. In this case, in the example illustrated in FIG. 7, the candidate agents that are therapeutic agents are represented by “Drug 1”, “Drug 2”, and “Drug 3”, and the effects of the candidate agents “Drug 1”, “Drug 2”, and “Drug 3” are represented by numerical values of priorities “1”, “2”, and “3”, respectively. Here, the higher the numerical values of the priorities, the higher the effects of the candidate agents.

In the gene therapy information, the ancillary information includes drug prices of the candidate agents, information on insurance to which the candidate agents are applicable, and information on clinical trials to which the candidate agents are applicable. In this case, in the example illustrated in FIG. 7, the drug prices of the candidate agents “Drug 1”, “Drug 2”, and “Drug 3” are represented by “100 (ten thousand yen)”, “50 (ten thousand yen)”, and “30 (ten thousand yen)”, respectively.

In addition, in the example illustrated in FIG. 7, in a case where the candidate agents “Drug 1” and “Drug 2” are used as therapeutic agents for the patient, a drug price is subsidized by 70° with respect to the drug price of the candidate agent “Drug 1” by application of the insurance under the public medical insurance system. In a case where the candidate agent “Drug 2” is used as a therapeutic agent for the patient, a drug price is subsidized by 30% with respect to the drug price of the candidate agent “Drug 2” by application of the “insurance A”. In addition, the candidate agent “Drug 3” is a clinical trial agent, and a cost required for the clinical trial of the patient is represented by “10 (ten thousand yen)”. In a case where the clinical trial is conducted at a different hospital from the patient's hospital, the clinical trial cost includes expenses such as costs for transportation to the hospital.

In this case, the candidate agent “Drug 1” is the least effective of the candidate agents “Drug 1”, “Drug 2”, and “Drug 3”, but the payment amount (300,000 yen) is within the budget of the patient. Therefore, the candidate agent “Drug 1” has the first priority order. In addition, although the payment amount (350,000 yen) of the candidate agent “Drug 2” is not within the budget of the patient, the candidate agent “Drug 2” is the next most effective after the candidate agent “Drug 1”. Therefore, the candidate agent “Drug 2” has the second priority order. Although the candidate agent “Drug 3” is the most effective of the candidate agents “Drug 1”, “Drug 2”, and “Drug 3”, the payment amount (400,000 yen) is not within the budget of the patient. Therefore, the candidate agent “Drug 3” has the third priority order.

Here, it is assumed that the budget of the patient as the patient information stored in the patient information DB 141 is changed. For example, as illustrated in (1) of FIG. 7, it is assumed that the budget of the patient is changed from “30 (ten thousand yen)” to “50 (ten thousand yen)”. The control function 152 redetermines the priority orders of the candidate agents based on a budget of the patient after being changed.

For example, as illustrated in (2) of FIG. 7, since the candidate agent “Drug 3” is the most effective of the candidate agents “Drug 1”, “Drug 2”, and “Drug 3”, and the payment amount (400,000 yen) is within the budget of the patient, the candidate agent “Drug 3” has the first priority order. In addition, since the candidate agent “Drug 2” is the next most effective after the candidate agent “Drug 3”, and the payment amount (350,000 yen) is within the budget of the patient, the candidate agent “Drug 2” has the second priority order. Since the candidate agent “Drug 1” is the next most effective after the candidate agent “Drug 2”, and the payment amount (300,000 yen) is within the budget of the patient, the candidate agent “Drug 1” has the third priority order.

In this case, the control function 152 generates the support information 420 in which the determined priority orders are associated with the genetic map 400 stored in the gene mechanism information DB 143. The presentation function 153 then presents to the doctor and the patient the support information 420 and the order table 430 representing the priority orders of the candidate agents “Drug 1”, “Drug 2”, and “Drug 3” by displaying the support information 420 and the order table 430 on the display of the terminal 50 of the doctor. For example, as illustrated in (3) of FIG. 7, the presentation function 153 presents to the doctor and the patient the order table 430 representing the priority orders of the candidate agents “Drug 1”, “Drug 2”, and “Drug 3”.

For example, in a case where the support information 420 is displayed on the display of the terminal 50, the presentation function 153 presents the priority orders at positions where the candidate agents act in the genetic map 400. In this case, in the example illustrated in FIG. 7, “(1)” indicating the first priority order is displayed on a position where the candidate agent “Drug 3” acts in the genetic map 400, and “(2)” indicating the second priority order is displayed on a position where the candidate agent “Drug 2” acts in the genetic map 400.

In addition, in a case where the support information 420 is displayed on the display of the terminal 50, the presentation function 153 presents the reasons for determining the priority orders of the candidate agents. Specifically, the presentation function 153 presents the priority orders of the candidate agents and the reasons by displaying first and second comments in the genetic map 400 on the display of the terminal 50. In this case, for the example illustrated in FIG. 7, the first comment indicates the position where the candidate agent “Drug 3” acts in the genetic map 400, and “Priority 1: Drug 3 is effective and the payment amount is within the budget (40)” is displayed as the first comment. In addition, the second comment indicates the position where the candidate agent “Drug 2” acts in the genetic map 400, and “Priority 2: Drug 2 is the next most effective after Drug 3 and the payment amount is within the budget (35)” is displayed as the second comment.

As described above, the medical information processing apparatus 100 according to the third embodiment receives the change in the patient information, and redetermines the priority orders of the candidate agents based on the information after the change is received, so that the patient can select the candidate agent according to the patient's desire. Therefore, by using the medical information processing apparatus 100 according to the third embodiment, treatment can be carried out with the candidate agent according to the patient's desire, so that it is possible to propose the treatment according to the patient's request.

### Fourth Embodiment

For example, in the medical information processing apparatus 100 according to a fourth embodiment, the ancillary information in the gene therapy information stored in the gene therapy information DB 144 further includes an adoption rate representing a percentage of adoption of each candidate agent. In this case, the control function 152 further determines the priority orders of the candidate agents based on the adoption rates of the candidate agents.

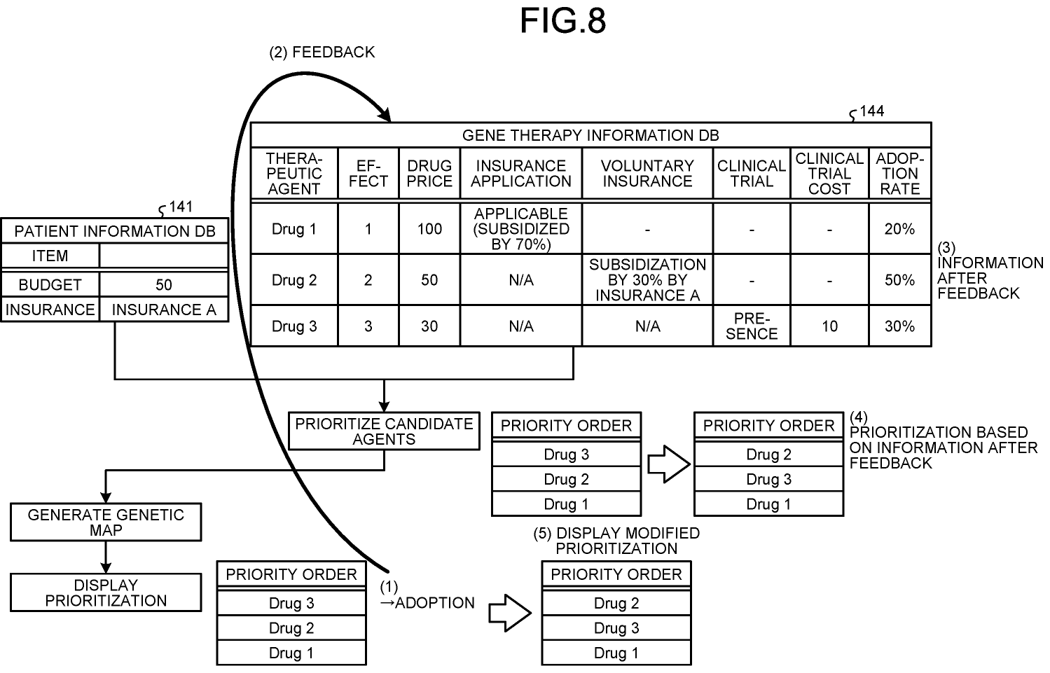

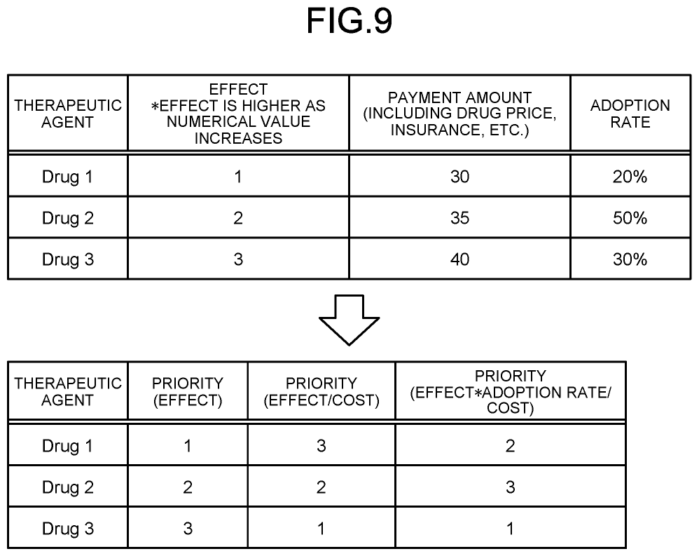

FIG. 8 and FIG. 9 are diagrams illustrating processing executed by the medical information processing apparatus 100 according to the fourth embodiment.

Regarding the medical information processing apparatus 100 according to the fourth embodiment, differences from the third embodiment will be described. In this case, as illustrated in (1) of FIG. 8, since the candidate agent “Drug 3” is the most effective of the candidate agents “Drug 1”, “Drug 2”, and “Drug 3”, and the payment amount (400,000 yen) is within the budget of the patient, the candidate agent “Drug 3” has the first priority order. In addition, for example, since the candidate agent “Drug 2” is the next most effective after the candidate agent “Drug 3”, and the payment amount (350,000 yen) is within the budget of the patient, the candidate agent “Drug 2” has the second priority order. Since the candidate agent “Drug 1” is the next most effective after the candidate agent “Drug 2”, and the payment amount (300,000 yen) is within the budget of the patient, the candidate agent “Drug 1” has the third priority order.

In addition, in the example illustrated in FIG. 8, in the gene therapy information, the ancillary information further includes the adoption rate representing a percentage of adoption of each candidate agent. The adoption rate is determined based on the frequency with which the candidate agent is selected. For example, the control function 152 receives candidate agents selected by each patient from the terminal 50 of the doctor, and determines the adoption rates of the candidate agents based on the received candidate agents. That is, the adoption rates of the candidate agent are updated whenever each candidate agent is selected.

For example, as illustrated in (2) of FIG. 8, the updated information is fed back to the gene therapy information DB 144, and as illustrated in (3) of FIG. 8, the adoption rates of the candidate agents “Drug 1”, “Drug 2”, and “Drug 3” are updated to “20%”, “50%”, and “30%”, respectively. In this case, as illustrated in (4) of FIG. 8, the control function 152 further determines the priority orders of the candidate agents based on the adoption rates of the candidate agents as the gene therapy information stored in the gene therapy information DB 144.

As illustrated in FIG. 9, the control function 152 calculates the effects (priority) of the candidate agents as follows: Effect×Adoption rate/Payment amount. For example, since the effect (priority) of the candidate agent “Drug 1” represents “1”, the payment amount is 30 (ten thousand yen), and the adoption rate is “20%”, the control function 152 calculates 1×20/30=0.66 as the effect (priority) of the candidate agent “Drug 1” in consideration of the adoption rate. In addition, since the effect (priority) of the candidate agent “Drug 2” represents “2”, the payment amount is 35 (ten thousand yen), and the adoption rate is “50%”, the control function 152 calculates 2×50/35=2.85 as the effect (priority) of the candidate agent “Drug 2” in consideration of the adoption rate. In addition, since the effect (priority) of the candidate agent “Drug 3” represents “3”, the payment amount is 40 (ten thousand yen), and the adoption rate is “30%”, the control function 152 calculates 3×30/40=2.25 as the effect (priority) of the candidate agent “Drug 3” in consideration of the adoption rate.

In this case, since the candidate agent “Drug 2” has the highest priority of the candidate agents “Drug 1”, “Drug 2”, and “Drug 3”, the candidate agent “Drug 2” has the first priority order. In addition, since the candidate agent “Drug 1” has the next highest priority after the candidate agent “Drug 2”, the candidate agent “Drug 1” has the second priority order. Since the candidate agent “Drug 3” has the next highest priority after the candidate agent “Drug 1”, the candidate agent “Drug 3” has the third priority order.

The control function 152 generates the support information 420 in which the determined priority orders are associated with the genetic map 400 stored in the gene mechanism information DB 143. The presentation function 153 then presents to the doctor and the patient the support information 420 and the order table 430 representing the priority orders of the candidate agents “Drug 1”, “Drug 2”, and “Drug 3” by displaying the support information 420 and the order table 430 on the display of the terminal 50 of the doctor. For example, as illustrated in (5) of FIG. 8, the presentation function 153 presents to the doctor and the patient the order table 430 representing the priority orders of the candidate agents “Drug 1”, “Drug 2”, and “Drug 3”.

For example, in a case where the support information 420 is displayed on the display of the terminal 50, the presentation function 153 presents the priority orders at positions where the candidate agents act in the genetic map 400. In this case, in the example illustrated in FIG. 8, “(1)” indicating the first priority order is displayed on the position where the candidate agent “Drug 2” acts in the genetic map 400, and “(2)” indicating the second priority order is displayed on the position where the candidate agent “Drug 3” acts in the genetic map 400.

In addition, in a case where the support information 420 is displayed on the display of the terminal 50, the presentation function 153 presents the reasons for determining the priority orders of the candidate agents. Specifically, the presentation function 153 presents the priority orders of the candidate agents and the reasons by displaying first and second comments in the genetic map 400 on the display of the terminal 50. In this case, for the example illustrated in FIG. 8, the first comment indicates the position where the candidate agent “Drug 2” acts in the genetic map 400, and “Priority 1: Drug 2 has a high priority (effect and adoption rate) and the payment amount is within the budget (35)” is displayed as the first comment. In addition, the second comment indicates the position where the candidate agent “Drug 3” acts in the genetic map 400, and “Priority 2: Drug 3 has the next highest priority (effect and adoption rate) after Drug 2 and the payment amount is within the budget (40)” is displayed as the second comment.

As described above, the medical information processing apparatus 100 according to the fourth embodiment further determines the priority orders of the candidate agents based on the adoption rate representing the percentage of adoption of each candidate agent, so that the patient can select the candidate agent according to the patient's desire. Therefore, by using the medical information processing apparatus 100 according to the fourth embodiment, treatment can be carried out with the candidate agent according to the patient's desire, so that it is possible to propose the treatment according to the patient's request.

The adoption rate is determined based on the frequency with which the candidate agent is selected, but the present embodiment is not limited thereto. For example, the adoption rate is determined based on the frequency with which the candidate agent is selected and new findings regarding the candidate agent. For example, the control function 152 receives the candidate agent selected by each patient from the terminal 50 of the doctor, and determines the adoption rate of the candidate agent based on the received candidate agent. That is, the adoption rate of the candidate agent is determined based on the frequency with which the candidate agent is selected and new findings regarding the candidate agent.

For example, new findings regarding the candidate agent include information such as candidate agents selected in the past with a budget similar to the budget of the patient, and candidate agents adopted under a condition of insurance purchase.

Specifically, it is assumed that, for example, the candidate agents selected in the past with a budget similar to the budget of the patient are the first and second candidate agents, and as a result of calculating the effects (priority) of the first and second candidate agents in consideration of the adoption rates, the first candidate agent has the higher priority than the second candidate agent. In this case, the control function 152 determines the priority orders of the candidate agents by prioritizing the priorities such that the priority of the first candidate agent is set to be higher than the priority of the second candidate agent, and the presentation function 153 presents the priority orders in association with the information representing the relationship between genes.

Similarly, it is assumed that the candidate agents adopted under a condition of insurance purchase are the first and second candidate agents, and as a result of calculating the effects (priority) of the first and second candidate agents in consideration of the adoption rates, the first candidate agent has the higher priority than the second candidate agent. In this case, the control function 152 determines the priority orders of the candidate agents by prioritizing the priorities such that the priority of the first candidate agent is set to be higher than the priority of the second candidate agent, and the presentation function 153 presents the priority orders in association with the information representing the relationship between genes.

For example, new findings regarding the candidate agent include information on the side effects of the candidate agents.

Specifically, it is assumed that, for example, the candidate agents are the first and second candidate agents, and as a result of calculating the effects (priority) of the first and second candidate agents in consideration of the adoption rate, the first candidate agent has the higher priority than the second candidate agent. It is further assumed that in a case of using the first candidate agent for a gene mutation Z, side effects occur, but from past results, the side effects occur to only a patient having both gene mutations Z and Y. On the other hand, it is assumed that no side effects occur to a patient who has only the gene mutation Z and a patient who has gene mutations Z and X even though the first candidate agent is used. For example, in a case where a patient subjected to treatment is the patient who has the gene mutations Z and X, the control function 152 determines the priority orders of the candidate agents by prioritizing the priorities such that the priority of the second candidate agent is set to be higher than the priority of the first candidate agent, and the presentation function 153 presents the priority orders in association with the information representing the relationship between genes.

In addition, it is assumed that, for example, the candidate agents are the first and second candidate agents, and as a result of calculating the effects (priority) of the first and second candidate agents in consideration of the adoption rate, the second candidate agent has the higher priority than the first candidate agent. In a case of using the second candidate agent, there are no side effects in literatures, for example, but from past results, it is assumed that there is a case where the side effects occur. In this case, the control function 152 determines the priority orders of the candidate agents by prioritizing the priorities such that the priority of the first candidate agent is set to be higher than the priority of the second candidate agent, and the presentation function 153 presents the priority orders in association with the information representing the relationship between genes. In this case, the presentation function 153 presents that the second candidate agent has the side effects.

The components of the devices illustrated in the present embodiment are functional conceptual components and are not necessarily physically configured as illustrated in the figures. That is, the specific form of distribution or integration of the devices is not limited to the form illustrated in the figures, and all, some, or one of the devices can be functionally or physically distributed or integrated in any unit according to various loads or usage conditions. Furthermore, all, some, or one of the processing functions performed by the devices may be implemented by a CPU and a computer program that is analyzed and executed by the CPU, or may be implemented as hardware with wired logic.

In addition, the method described in the present embodiment can be implemented by executing a computer program prepared in advance on a computer such as a personal computer or a workstation. This computer program can be distributed via a network such as the Internet. In addition, this computer program is recorded on a non-transitory computer readable recording medium such as a hard disk, flexible disk (FD), CD-ROM, MO, or DVD, and executed by being read out from the recording medium with a computer.

According to at least one embodiment described above, it is possible to propose treatment according to the patient's request.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry, wherein
the processing circuitry is configured to
acquire gene therapy information in which a candidate agent that is a therapeutic agent for a gene mutation related to a disease of a patient, an effect of the candidate agent, and ancillary information on the candidate agent are associated with one another;
determine a priority order of the candidate agent based on the gene therapy information and patient information on the patient; and
present the priority order in association with information representing a relationship between genes.

2. The medical information processing apparatus according to claim 1, wherein
the information representing the relationship is a correlation diagram illustrating a relationship between genes, and
the processing circuitry is configured to present the priority order at a position where the candidate agent acts in the correlation diagram.

3. The medical information processing apparatus according to claim 1, wherein the processing circuitry is configured to present a reason for determining the priority order.

4. The medical information processing apparatus according to claim 1, wherein
the patient information includes a budget of the patient; and
the ancillary information includes a drug price of the candidate agent.

5. The medical information processing apparatus according to claim 1, wherein
the patient information includes information on insurance that the patient has taken out; and
the ancillary information includes information on insurance to which the candidate agent is applicable.

6. The medical information processing apparatus according to claim 1, wherein the ancillary information includes information on whether the candidate agent is a clinical trial agent, and further includes a cost required for a clinical trial in a case where the candidate agent is the clinical trial agent.

7. The medical information processing apparatus according to claim 1, wherein
the patient information includes information on a level of living desired by the patient; and
the ancillary information includes information on a side effect of the candidate agent.

8. The medical information processing apparatus according to claim 1, wherein
the patient information includes a medication history of the patient; and
the ancillary information includes information on a component contained in the candidate agent.

9. The medical information processing apparatus according to claim 1, wherein the processing circuitry is configured to receive a change in the patient information and redetermine the priority order of the candidate agent based on information after the change is received.

10. The medical information processing apparatus according to claim 1, wherein
the ancillary information further includes an adoption rate representing a percentage of the candidate agent adopted; and
the processing circuitry is further configured to determine the priority order of the candidate agent based on the adoption rate of the candidate agent.

11. The medical information processing apparatus according to claim 10, wherein the adoption rate is determined based on frequency with which the candidate agent is selected and new findings regarding the candidate agent.
